(19) United States
(12) Patent Application Publication
(10) Pub. No.: US 2018/0291325 A1
(43) Pub. Date: Oct. 11, 2018

(54) IN VITRO PHARMACOKINETIC-PHARMACODYNAMIC DEVICE

(71) Applicant: Merck Sharp & Dohme Corp., Rahway, NJ (US)

(72) Inventors: Donna M. Carr, Kenilworth, NJ (US); Michael K. Wismer, Lebanon, NJ (US); Charles G. Garlisi, Hamilton Square, NJ (US)

(73) Assignee: Merck Sharp & Dohme Corp., Rahway, NJ (US)

(21) Appl. No.: 15/573,981

(22) PCT Filed: May 9, 2016
(86) PCT No.: PCT/US16/31400

§ 371 Date: Nov. 14, 2017

Related U.S. Application Data
(60) Provisional application No. 62/161,489, filed on May 14, 2015.

(51) Int. Cl. C12M 3/06; C12M 1/12; C12M 1/00; C12M 1/36; G01N 33/50

(52) U.S. Cl. CPC C12M 27/16; C12M 23/06; C12M 25/02; G01N 33/5008; C12M 37/02; C12M 25/16; C12M 41/48; C12M 29/14

(57) ABSTRACT

The present invention provides an in vitro cell culturing bioreactor device capable of supporting the growth of adherent or non-adherent mammalian cells. The invention also provides methods for monitoring and/or measuring the effects of therapeutic agents on cells, for determining the pharmacokinetic-pharmacodynamic relationship between a drug and a target cell, and for determining an effective dosing regimen for a drug or therapeutic agent.

### FIELD OF THE INVENTION

The present invention provides an in vitro low-volume cell culturing bioreactor device capable of supporting the growth of adherent or non-adherent mammalian cells. The invention also provides methods for monitoring and/or measuring the effects of therapeutic agents on cells and for determining the pharmacokinetic-pharmacodynamic relationship between a drug or drug candidate and a target cell.

### CROSS-REFERENCE TO RELATED APPLICATIONS

This application claims the benefit of U.S. Provisional Application No. 62/161,489, filed May 14, 2015, which is herein incorporated by reference in its entirety.

### BACKGROUND OF THE INVENTION

In vitro PK/PD (pharmacokinetic/pharmacodynamic) systems have been developed in order to study cellular response under drug dosing conditions that mimic those seen in vivo. These systems enable predictive studies to bridge between preclinical data and the results seen from clinical trials. They can be used, for example, to study the emergence of resistant mutants during dosing, determine PK drivers and to study tachyphylaxis responses. Examples of these in vitro PK/PD systems include those based on hollow-fiber technology and the BelloCell® High Density Cell Culture System (Cesco BioProducts, Atlanta, Ga.). Hollow fiber bioreactor technology and the BelloCell® were developed as an economical alternative to traditional methods for cell culturing. Cell culture requires periodic splitting as the surface area of the culture dishes is limited and the levels of waste, pH and nutrients are in constant flux.

Hollow-fiber bioreactors can result in higher cell densities than with traditional cell culture methods and through the use of a semi-permeable membrane to retain high molecular weight proteins in the extra-capillary space allow for more efficient use of costly medium components. Thus, hollow fiber bioreactors have frequently complemented animal models in the study of the pharmacokinetic and pharmacodynamic relationship between a therapeutic agent and the administration thereof. Such in vitro studies are advantageous because they provide important pre-clinical data, which can be evaluated before investing the time and expense needed to carry out clinical studies. In vitro studies can allow a researcher or drug manufacturer to more quickly and economically test a variety of approaches and dosages than would be possible with animal models alone. Examples of such in vitro systems for evaluating the effects of drugs or therapeutic agents on cells cultured in hollow fiber bioreactors are described in U.S. Pat. Nos. 4,391,912; 5,622,857; 5,962,317; 6,001,585; 6,287,848 and 6,670,169, and Wang et al., 2008, J Antimicrobial Chemotherapy 62:1070-1077.

The BelloCell® employs bellows built into the culture bottle which compress and expand to optimize oxygenation and allowing optimal adherence to an appropriate substrate. The BelloCell® technology is described in U.S. Pat. No. 7,033,823. The device consists of a hollow cylinder in which a porous, fibrous matrix is located between an upper and a lower basket, the matrix serving as a bedding for the cells. An upper chamber is situated above, and a lower chamber below the bedding matrix. The lower chamber essentially consists of a compressible bellows-type bag, by means of which liquid cell growth medium can be recirculated to the upper chamber. An example of an in vitro system for evaluating pharmacodynamics of a drug or therapeutic agent on cells cultured in BelloCell® bioreactor is described in Brown et al., 2012, Antimicrobial Agents and Chemotherapy 56:1170-1181.

### SUMMARY OF THE INVENTION

Provided herein is an in vitro cell culture system comprising (a) a central compartment (e.g., a 50 ml LeviTube™) comprising cell culture medium and a filter; (b) a first syringe pump for supplying cell culture medium to the central compartment, wherein the first syringe pump has a unidirectional valve and wherein the inlet line of the valve on the syringe pump is connected to the media vessel and the outlet line of the valve on the syringe pump is connected to the central compartment; (c) one or more additional syringe pumps for supplying one or more test drugs attached to the central compartment via one or more additional connection lines; and (d) a waste syringe pump used to remove waste products and drugs from the central compartment, wherein the waste syringe pump has a unidirectional valve and wherein the inlet line of the valve on the waste syringe pump is connected to the central compartment and the outlet line of the valve on the waste syringe pump is connected to the waste vessel; wherein the central compartment can be oscillated from about 180° to about 360° in both directions on a single plane and wherein the oscillations occur as a net zero oscillating periodic function to prevent twisting of the media and drug delivery lines around each other. In certain embodiments of the invention, the filter prevents cells from entering the waste media (second connection) line. In certain aspects of this embodiment, particularly for non-adherent cells, the filter is a hollow-fiber filtration tip.

A key feature of the invention is the oscillation of the central compartment to achieve a low-shearing environment for cell growth. It is important that the central compartment be oscillated around a vertical axis such that the movement in one direction is counteracted by an equal movement in the opposite direction. The oscillations can be from 180 degrees to 360 degrees in both directions but must occur in a net zero oscillating periodic function and performed in such a manner such that the cumulative effect over time does not result in the twisting of the media and drug delivery lines around each other. The rate of oscillations can be adjusted from <1 oscillation/minute to >120 oscillations/minute based on the shear tolerance of the cell line being used. In one embodiment, one oscillation occurs every 2 seconds. In certain embodiments of the invention, the oscillation is achieved using a programmable motor.

In certain embodiments of the invention, the in vitro cell culture system provides a low shear cell culture environment. In certain aspects of this embodiment, the low shear cell culture environment is provided by a central compartment which is a vial having between 2 and 10 baffles along the interior side of the vial wall. In certain subaspects, the vial has between 2 and 6 baffles.

In certain embodiments of the invention, the vial has a cap. In certain aspects of this embodiment, the cap has a port for introducing and removing cell culture media, one or more ports for introducing one or more drugs, one port for removal of cell media and media samples from the central compartment and a venting port.

In certain embodiments of the invention, the syringe pumps are controlled by computer software that controls pump rates and infusion times.

In certain embodiments of the invention, the in vitro cell culture system further comprises a substrate for adherent cells to grow. In certain aspects of this embodiment, the substrate is a microcarrier or plastic flake. In certain sub-aspects, the substrate is a microcarrier composed of collagen-, fibronectin-, pronectin-, gelatin-coated or uncoated microbeads. In certain aspects of this embodiment, the filter is a porous polyvinylidene fluoride substrate or a porous stainless steel substrate with pore size less than 100 microns. In certain aspects of this embodiment, the filter is a tip adapter with hollow fibers consisting of 0.2 μm hydrophilic mixed cellulose ester hollow fibers.

The present invention is also directed to methods of using the in vitro cell culture system described herein, the method comprising the steps of: a) seeding 1×10⁶ to 5×10⁷ cells in the central compartment; b) placing the central compartment in connection with the in vitro culture system described herein; c) exposing the cultured cells to one or more drugs; and d) removing samples of culture medium for testing; and/or removing samples of the cells for testing. The methods of the invention can be used in situations where the amount of the one or more drugs is added by one or more bolus infusions, is added periodically, is constant over time, or is added at varying rates.

In certain embodiments of the invention, the methods further comprise using mathematical modeling to achieve one or more pharmacokinetic parameters selected from: maximum concentration (Cₘₐₓ), the area under the drug concentration-time curve (AUC), the time of peak drug concentration (Tₘₐₓ), the clearance rate (Cl), the drug elimination rate (volume per unit time), the concentration before the next administered dose (Cₘᵢₙ), the drug half-life (T_{1/2}) or any combination thereof. In certain aspects of this embodiment, the method further comprises testing the removed cell sample for a pharmacodynamics property selected from cell viability, cell growth, cell shape, viral load, expression of a protein, post-translational modification of a protein, DNA content, modification of DNA, RNA content, expression of a lipid marker or any combination thereof.

The present invention is also directed to methods of determining the relationship between a pharmacokinetic parameter of a test drug and a pharmacodynamic effect of the test drug on target cells, comprising: (a) growing the cells in the central compartment of the in vitro culture system described herein; (b) contacting the cells with at least one test drug; (c) adjusting the in vitro culture system to establish one or more pharmacokinetic parameters; (d) measuring at least one pharmacodynamic effect of the test drug on target cells; and (e) determining a result.

### BRIEF DESCRIPTION OF THE DRAWINGS

FIG. 1 is a schematic diagram of the PK/PD cell culture system of the invention. Cells of interest can be grown on microcarriers suspended within a central compartment (e.g., 50 ml LeviTube™) in an oscillating flow-through system that allows for homogeneous exposure of cells to drug/drug metabolite solutions.

FIGS. 2A-B are diagrams of the central compartment assembled (A) and disassembled (B) including the culture tube, tube holder and motor for oscillation. Reference numerals indicate the following parts: (1) motor cover, (2) motor, (3) magnetic coupling, (4) tube holder, (5) culture tube, (6) tube cap, (7) dip tube, (8) filter, (9) tee, (10) inlet tube, and (11) outlet tube.

FIG. 3 is a diagram of an exemplary cap of the invention with tapped ¼-28 ports for connection of 1/16″ tubing (fluid and vent connections) and 8-32 tapped ports around the perimeter of the cap for extended point set screws to secure the cap to a LeviTube. FIG. 3A shows the outside of the cap, which is shown as transparent for clarity. FIG. 3B is a top view of the cap, which shows the location of four ¼-28 screw ports (screw diameter ¼ inch, 28 threads/inch). Distance L1 corresponds to the diameter of the threaded section of the screw port (0.213″), distance L2 corresponds to the diameter of the cap (1.565″), and distance L3 corresponds to the depth of the screw port (0.350″), with 0.67″ holes being drilled through the top of the cap (see side view in FIG. 3D). FIG. 3C shows a side view of the cap with distance L4 corresponding to the height of the cap (0.950 inches). FIG. 3D also shows a side view of the cap, with distance L5 corresponding to the diameter of the holes drilled in the side of the cap to hold retaining screws 120° apart.

FIG. 4 is a graph of the concentrations of a HCV NS3/4 protease inhibitor (Compound) (PK) achieved in the central compartment of the device (blue diamonds) compared to the targeted concentrations (solid line) over three QD (once daily) infusions described in Example 2.

FIG. 5 represents the drop in HCV genotype 1B viral RNA compared to pretreatment controls (PD) as determined by reverse transcription polymerase chain reaction (RT-PCR) assay of samples removed from the central compartment over three QD infusions of Compound described in Example 2.

FIGS. 6A-B represent the drop in HCV genotype 1B RNA compared to pretreatment controls (PD) as determined by A) Luciferase reporter assay; or B) RT-PCR assay; of samples removed from the central compartment over three QD infusions of Compound targeting 4 different AUC concentrations (dose escalation) as described in Example 3.

FIG. 7 is a graph of the actual concentrations of Compound (PK) achieved in a single experiment utilizing 4 devices. As described in Example 3; three QD infusions, targeting 0.3×, 1×, 3× or 10× the AUC for Compound of 16.8 nM (dose escalation), were delivered to four devices simultaneously and samples were removed for PK determination over time (solid lines and symbols). Targeted concentrations of Compound are represented by dashed lines.

FIGS. 8A-B represent the drop in HCV genotype 1B RNA compared to pretreatment controls (PD) for the Compound dose fractionation experiment described in Example 3; Decrease in viral load was determined by A) Luciferase reporter assay; or B) RT-PCR assay. Samples were removed once daily from the central compartments for PD analysis. Compound was delivered to 4 devices as 1) a constant 72 hr infusion; 2) 3×QD infusions with each AUC targeted at 16.8 nM; 3) 6×BID (twice daily) infusions with each AUC targeted at 8.4 nM 4) 12×TID (three times daily) infusions with each AUC targeted at 5.6 nM.

### DETAILED DESCRIPTION OF THE INVENTION

The in vitro cell culture system described herein provides a cell culture environment that keeps adherent cells on microcarriers or non-adherent cells in suspension. Experiments performed in prior systems, such as hollow-fiber systems or the BelloCell®, using translational PK/PD studies with adherent mammalian cells utilize large volumes of cells, compounds, serum and media. One drawback of a BelloCell® device is that during the expansion of the bellows-type bag, a vacuum is generated, which causes a cell-shearing action, which is detrimental to the cultivation of cells. Furthermore, the collection of cellular material for PD marker analysis from these devices is cumbersome. The present invention provides a system where cells can be maintained in suspension by oscillation without the twisting of the tubing required for media delivery and removal. In addition, the simplified operation of the in vitro PK-PD system described herein compared to the BelloCell® or hollow fiber systems allows for thorough in vitro characterization of drugs under conditions that simulate human pharmacokinetics (PK based on data from clinical trials). By employing well-defined conditions which allow for investigation of individual factors impacting the PD in an in vitro system, the number of animal studies which need to be conducted can be decreased ultimately saving time, money and animals.

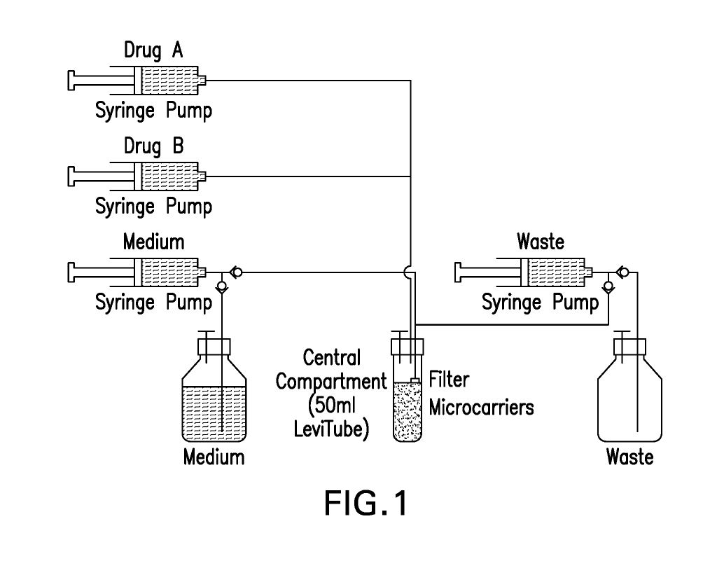

Shown in FIG. 1 is a HulaCell cell culture system that is based on a disposable test tube (shown with optional microcarriers) and a modified cap to permit the addition of an inlet/outlet tube (for media and drugs), an inlet port for drug infusion, a sampling port, and a venting port. This HulaCell cell culture system can be used to define in vitro the pharmacodynamics (PD) and pharmacokinetics (PK) of drugs in non-adherent and adherent cells. The system, like the Bellocell® and hollow-fiber systems, can support translation from in vitro to animal models and human clinical trials. However, the HulaCell employs reagent volumes typically 10-12 fold less than the BelloCell®. For example, the BelloCell® typically employs a 500 ml bioreactor. In contrast, the HulaCell typically employs a 30 to 50 ml bioreactor which could be further reduced to as low as 10 ml if required.

Also provided herein are methods of using the in vitro cell culture systems of the invention by seeding adherent cells on microcarriers or non-adherent cells in a central compartment; placing the central compartment in connection with an in vitro culture system as described herein; exposing the cultured cells to one or more drugs; and d) removing samples of culture medium for testing and/or removing samples of the cells for testing. Such testing can include the relationship between the dose of a drug given to a patient (pharmacokinetics: PK) and the utility of that drug in treating the patient's disease (pharmacodynamics: PD). In one embodiment, the in vitro cell culture systems can be used for integrated PK-PD analyses of drugs and for testing the toxicity of drugs of interest.

Pharmacokinetics concerns the absorption, distribution, biotransformation (metabolism, if it exists), and the elimination of drugs, i.e., what the body does to the drug. Relevant pharmacokinetic terms include Cₘₐₓ, the peak (maximum) concentration; AUC, the area under the drug concentration-time curve; Tₘₐₓ, the time of peak drug concentration; Cl (clearance rate), the measure of the body's ability to eliminate the drug (volume per unit time); volume of distribution, the ratio of the amount of drug in the body to the concentration of the drug in the blood; and Cₘᵢₙ, the concentration before the next dose is administered.

Pharmacodynamics is the study of the relationship between drug concentration and intensity of pharmacological effect, i.e., what the drug does to the body or target cell. Relevant pharmacodynamic terms include Eₘₐₓ, the maximum effect, ED₅₀, the dose which produces 50% of the maximum effect, and EC₅₀, the concentration observed at half the maximal effect, or any other possible measurable change in a characteristic or phenotype of the target cell.

As used herein, the term “drug” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such drugs may render it suitable as a “therapeutic agent”, which is a biologically, physiologically, or pharmacologically active substance (or substances) that acts locally or systemically in a subject. By drug, it also meant a drug candidate or compound to be tested for biological, physiologic or pharmacologic activity.

As used herein, the term “mammal” includes humans, primates, bovines, porcines, canines, felines, and rodents (e.g., mice and/or rats).

As used herein, the phrase “net zero oscillating periodic function” refers to an oscillating pattern where the net movement of a fixed point on the tube is zero (i.e., it returns to the same position where it began) over a fixed time period or number of cycles. For example, the net zero oscillating periodic function can be over 1, 2, 5, 10, 15, 20, 30 seconds, or 1, 2, 3, 4, or 5 minutes. The net zero oscillating periodic function can also be over every cycle (i.e., for an oscillation in one direction, there is an equal movement in the opposite direction), or 2, 3, 4, 5, 6. 7, 8, 9, 10, 15, 20, 25, 30, 35, 40, 45, 50, 60, 70, 80, 90, 100, 125, 150, 175, 200, 250 or 300 cycles.

As used herein, the terms “patient,” “individual,” “subject” or “host” refers to either a human or a non-human animal.

As used herein, the term “therapeutic agent” refers to any chemical moiety that is a biologically, physiologically, or pharmacologically active substance that acts locally or systemically in a subject. This term also refers to any substance intended for use in the diagnosis, cure, mitigation, treatment or prevention of disease or in the enhancement of desirable physical or mental development and/or conditions in an animal or human. For example, the therapeutic agent may be any of the drugs described herein.

As used herein, the term “therapeutic effect” refers to a local or systemic effect in animals, particularly mammals, and more particularly humans, caused by a pharmacologically active substance. The phrase “therapeutically effective amount” means that amount of such a substance that produces some desired local or systemic effect at a reasonable benefit/risk ratio and is applicable to any treatment. The therapeutically effective amount of such substance will vary depending upon the subject and disease or condition being treated, the weight and age of the subject, the severity of the disease or condition, the manner of administration and the like, which can readily be determined by one of ordinary skill in the art. For example, certain compositions described herein may be administered in a sufficient amount to produce a desired effect at a reasonable benefit/risk ratio applicable to such treatment.

### Central Compartment

The present invention relates, in part, to a central compartment which serves as a bioreactor for growing and culturing non-adherent or adherent mammalian cells in an in vitro environment. The central compartment comprises a suitable reservoir for cell culture.

While the central compartment is represented in FIG. 1 as a tube, any suitable shape (e.g., elongate, elliptical, spherical, etc.) may be utilized so long as a cap can be made for it. For example, the central compartment chamber can be cylindrical, box-like shaped, rectangular-like shaped, bag-like shaped, spherically shaped, and the like. Accordingly, the central compartment may be in the form of a vial, test tube, bottle, flask, bag, etc.

Any suitable reservoir for tissue culture may be used with no particular limitation on the materials that can be used to form the surface of the central compartment. Preferably, the material does not harm or is not toxic to the cells in the bioreactor. Examples of such materials include glass, acrylic resins, polycarbonate, polyolefin resins, polystyrene, silicone polymer, polysulfone, polyethylene, polyurethane, etc. For supplying sufficient amounts of oxygen to proliferating cells, materials having excellent gas permeability may be used to vent the central compartment, such as a thin silicone polymer film or tube, a thin silicone polymer film reinforced by a polyester mesh, a thin polybutadiene film or tube, a thin silicone-polycarbonate copolymer film or tube, a porous teflon film, a porous polypropylene film, etc., the thickness of which can be from 0.01 mm to 3 mm, 0.05 mm to 5 mm, or 1.0 to 10 mm, or any other suitable thickness.

While any volume of central compartment can be used that is suitable for tissue culture, a significant benefit of the present invention is the ability to use cell culture volumes smaller than those previously used. In particular, the volume of the central compartment is less than 500 mls, for example, 300 mls, 250 mls, 200 mls, 150 mls, 100 mls, or 50 mls. In one embodiment, the central compartment has a maximum capacity of 50 ml. In this embodiment, generally, 35-40 mls of media (including cells and, if used, microcarriers) is in the central compartment. Where the volume of media is less than the capacity of the central compartment, a portion of its internal volume is retained for a gas, such as oxygen, carbon dioxide, nitrogen or a mixture thereof, to assist in the aeration of cultures.

In certain embodiments of the invention, the in vitro cell culture system, particularly, the central compartment provides a low shear cell culture environment. In a preferred embodiment, the tube has baffles along the walls. Typically the central compartment has 6 baffles consisting of thin, plastic paddles that run vertically from the bottom of the tube. The paddles can vary in length from 1 to 6 cm but typically do not extend beyond the mid-point of the tube. These paddles extend perpendicularly mm from the wall of the tube and gently mix the contents of the tube without moving parts while avoiding unidirectional shear. In certain aspects of this embodiment, the low shear cell culture environment is provided by central compartment which is a vial having between 2 and 10 baffles along the interior side of the vial wall. In certain subaspects, the vial has between 2 and 6 baffles. A commercially available central compartment is the 50 ml LeviTube™.

The central compartment also comprises a filter. The purpose of the filter is to prevent cells and/or microcarriers from being removed from the central compartment into the waste line. Accordingly, the molecular weight cut off of the filter is appropriate to prevent cells and/or microcarriers to pass through the filter. The filter can have pores having a pore size of about 0.1 micron to about 150 micron. In certain aspects of this embodiment, the filter is a porous polyvinylidene fluoride substrate with pore size less than 100 microns. Suitable filters include, but are not limited to, hollow-fiber filtration tips (DynaGard® tips; Spectrum Labs), porous stainless steel with pore size less than 100 microns or glass fiber. In certain aspects of this embodiment, particularly for non-adherent cells, the filter is a hollow-fiber filtration tip.

In certain embodiments of the invention, the filter is provided on the end of a dipping tube which serves as a media inlet/outlet conduit. The dipping tube is typically a rigid or semi-rigid piece of tubing that can be stainless steel, TEFZEL® (ETFE: ethylene-tetrafluoroethylene) tubing, PTFE (polytetrafluoroethylene) tubing, PEEK (polyetheretherketone) tubing or any other chemically inert rigid or semi-rigid tubing of suitable outer diameter to fit through the ports in the HulaCell cap. The dipping tube can be inserted into the central compartment such that the filter at the end of the dipping tube is at the top of the media. Thus, while the waste syringe pump is typically of a higher volume, the dipping tube draws only air when the level of media is below the filter.

In another embodiment of the invention, the dipping tube can be inserted into the central compartment such that the filter at the end of the dipping tube is submerged in the media. In this embodiment the size of the waste syringe pump is typically the same as the size of the media syringe and the amount of media delivered is balanced by the amount of waste removed.

In another embodiment of the invention, the dipping tube can be inserted into the central compartment such that the end of the dipping tube is submerged in the media but the filter, a hollow fiber filtration tip, is at the top of the dipping tube on the external side of the cap. In this embodiment the size of the waste syringe pump is typically the same as the size of the media syringe and the amount of media delivered is balanced by the amount of waste removed.

A key feature of the invention is the oscillation of the central compartment to achieve a low-shearing environment for cell growth. It is important that the central compartment be oscillated around a vertical axis such that the movement in one direction is counteracted by an equal movement in the opposite direction. The oscillations can be from 180 degrees to 360 degrees in both directions but must occur as a net zero oscillating periodic function and performed in such a manner as to prevent distortion or twisting of the media and drug delivery lines around each other. The speed of the oscillations may be varied from <1 oscillation per minute to >120 oscillations per minute. In certain embodiments of the invention, the oscillation is achieved using a programmable motor. The motor is preferably programmable, and allows oscillation of the central tube. Precisely indexing motors are used to provide the bi-directional oscillation necessary to keep cell or microcarriers in suspension without twisting the tubing needed for media delivery and removal. Typical oscillation rates are 1/sec. The motor allows for complete rotation in both directions. The oscillation keeps the cells mixed into solution and allows proper aeration. The oscillation occurs in a reciprocating motion.

In certain embodiments of the invention, the vial has a cap. In certain aspects of this embodiment, the cap has a port for introducing and removing cell culture media, one or more ports for introducing one or more drugs, and a venting port. In certain embodiments of the invention, the cap has a first connection port, a second connection port, a venting port and optionally a sampling port, wherein the first connection port has a dip tube to which a filter is attached. Fresh media is delivered to the vial through the dip tube and waste is removed from the tube through the dip tube. The venting port allows gaseous cell waste products to be removed from the system.

Any suitable material for tissue culture may be used for the cap. Preferably, the material does not harm or is not toxic to the cells in the bioreactor. Examples of such materials include glass, acrylic resins, polycarbonate, polyolefin resins, polystyrene, silicone polymer, polysulfone, polyethylene, polyurethane, etc. In one embodiment, the cap is constructed of acetal resin (e.g., Delrin®). For supplying sufficient amounts of oxygen to proliferating cells, materials having excellent gas permeability may be used to cover the venting port, such as a thin cellulose acetate membrane or a thin surfactant free cellulose acetate membrane, the thickness of which can be from 50 μm to 200 μm or any other suitable thickness.

### Fluidics

The central compartment is coupled to an external fluid delivery system that comprises a media pump, e.g., a syringe pump, which delivers fresh media to the central compartment, a waste pump, e.g., a syringe pump, which draws spent media from the central compartment, and optionally one or more pumps, e.g., syringe pumps, for delivering drug(s). In one embodiment, the central compartment can be coupled to an external fluid delivery system having a media syringe pump, a waste syringe pump, and one or more syringe pumps for delivering drug(s).

In one aspect, the central compartment is coupled to an external fluid circulating system having (i) a first syringe pump for supplying fresh cell culture medium to the central compartment, wherein the first syringe pump has a unidirectional valve and wherein the inlet line of the valve on the syringe pump is connected to the media vessel and the outlet line of the valve on the syringe pump is connected to the central compartment; (ii) one or more additional syringe pumps for supplying one or more test drugs attached to the central compartment via a one or more additional connection lines; and (iii) a waste syringe pump used to remove waste products and drugs from the central compartment, wherein the waste syringe pump has a unidirectional valve and wherein the inlet line of the valve on the waste syringe pump is connected to the central compartment and the outlet line of the valve on the waste syringe pump is connected to the waste vessel.

Any known external fluid circulating system can be used in connection with the bioreactor of the invention. Particular examples of such systems are described in U.S. Pat. Nos. 3,883,393; 4,220,725; 4,144,136; 4,391,912; 4,889,812; 4,999,298; 5,290,700; 5,622,857; 5,763,261; 5,955,353; 5,962,317; 6,001,585; 6,287,848; and 7,270,996; and U.S. Published Application No. 2004/0029265.

In each case, the central compartment and cap can be modified to be used in conjunction with the above systems. The shape of the central compartment and the particular positioning of inlet, outlet and optional ports may be designed in various configurations such that the chamber is capable of being coupled via necessary tubing and the like (i.e., adapted to fit) to a suitable external fluid delivery system. The fluid delivery system preferably is one whereby one or more desired pharmacokinetic parameters can be set such that PK of a particular drug or therapeutic agent can be mimicked.

Syringe pumps, including reciprocating syringe pumps, are commercially available from sources including New Era Pump Systems, Inc., Farmingdale, N.Y. The media syringe pump typically employs a 1 ml syringe and can be kept at a constant flow rate. The waste syringe pump typically employs a 3 ml syringe. Preferably, the reciprocating cycle of the waste syringe pump is balanced with the media syringe pump through the use of a multi-channel reciprocating syringe pump in which the media and waste syringes for one central compartment are driven by the same multi-channel pump. In certain embodiments of the invention, the waste syringe pump is employed at a higher volume and flow rate to accommodate the addition of drug. The drug syringe(s) are controlled by separate, single-channel infusion pump(s). Typically a 5-20 ml syringe is used to infuse the drug solution.

The pumps may be controlled by custom computer software developed which enables the delivery and dilution of compounds in such a way as to create PK profiles. These profiles can mimic those seen in vivo or can be PK profiles that cannot be achieved in vivo in order to perform PK driver experiments. For example, one application uses VB.NET software running on a Windows PC (Microsoft Corporation, Redmond, Wash.). The software allows the user to set pump rates and infusion times at the start of the experiment. It also performs calculations to assist with setting media, waste, and infusion pump rates based on desired pharmacokinetic parameters. During the experiment, the program handles the timing of all commands issued to media, waste, and infusion pumps. In addition, the software allows the user to extend the simulated half-life of an infused drug by mathematically varying the rate of the infusion pump for that drug during the infusion.

The media bottle and waste bottle can be chosen from any standard cell culture container such as Nalgene™ bottles or Erlenmeyer flasks.

The tubing used in the systems of the invention is preferably inert, chemical resistant and non-porous. Suitable examples of such tubing include, but are not limited to, those made from silicone, vinyl including PVC, fluoropolymers including PFA (perfluoroalkoxy), FEP (fluorinated ethylene propylene) and PTFE (polytetrafluoroethylene), PVDF (polyvinylidene fluoride) and polyethylene. Suitable tubing is commercially available from sources including IDEX Health & Sci., Thermo Sci., etc.

In certain embodiments, samples of the media for PK determination may be removed through the connection tube leading to waste vessel. It would be possible to incorporate a diversion valve into the waste outlet line in order to collect larger volumes of drug/media for PK analysis. Typical flow rates in the HulaCell are ˜100-300 μl/min so sample collection could take 5-10 min. A fraction collector can also be incorporated.

Generally, the bioreactor of the present invention, when coupled with a suitable external fluid delivery system, can be used to grow cells and monitor their growth by sampling the media and/or cells from the central compartment and measuring or characterizing the cell growth based on various factors, such as, pH, metabolite composition, protein expression, gene expression, presence of extracellular expression products (e.g., certain enzymes etc.) and observable physical cell features or phenotypes.

Referring to FIG. 1, an embodiment of the HulaCell system is depicted in a schematic view. In this schematic, the system differs from a BelloCell® continuous cell culture system in the use of a central compartment based on a test tube rather than a BelloCell® bottle. In an exemplary embodiment, the test tube is impeller free and has baffles along the tube wall to allow mixing without shearing. The tube cap has multiple connector ports. As depicted in FIG. 1, in this embodiment, the tube cap has three connector ports. A first connector port has a dip tube below a T-fitting to which is connected a syringe pump for delivering media and a syringe pump for removal of waste. A second connection tube is for delivering the drug(s) and is attached to one or more syringe pumps for delivery of the drug(s). A third connector port is used for venting which regulates pressure and aeration. An optional fourth connector port can be used for sampling. A medium storage bottle is attached to the medium syringe pump. A waste bottle is attached to the waste syringe pump. Unidirectional valves permit the correct flow of media. Venting ports are also present on the medium storage bottle and waste bottle. The medium syringe pump and waste syringe pump are preferably controlled in unison as a reciprocating syringe pump to allow for the continual flow and circulation of fresh growth medium through the central compartment. Not depicted in FIG. 1 is a means to oscillate the central compartment.

Also not depicted in FIG. 1 is the optional port to enable the sampling of cells, media and/or cell products, the testing of various characteristics of the media (e.g., pH), or the adding of additional growth supplements and nutrients to the extra-membrane compartment. One or more additional ports may be employed in the test tube cap. In one embodiment, the sampling port is designed such that a 1 ml syringe can be attached to the sample port to remove media from the central compartment. Such design alterations can be made without undue experimentation. In the absence of this optional port, spent media can be sampled from the waste stream or cells and/or media can be removed from the culture tube by removing the cap and removing samples by, e.g., a pipette.

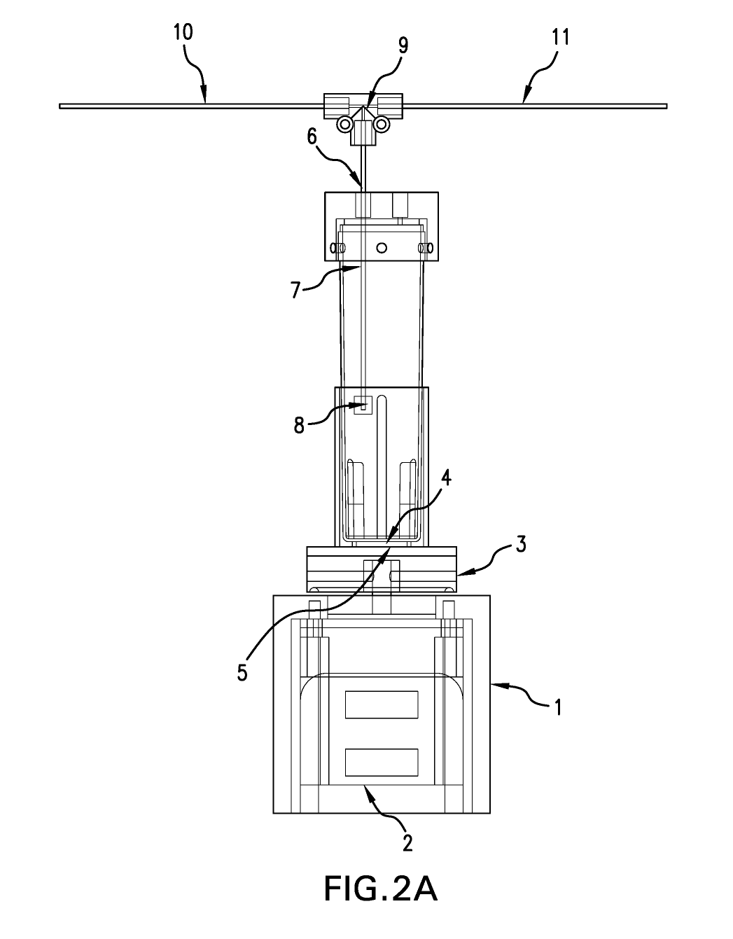

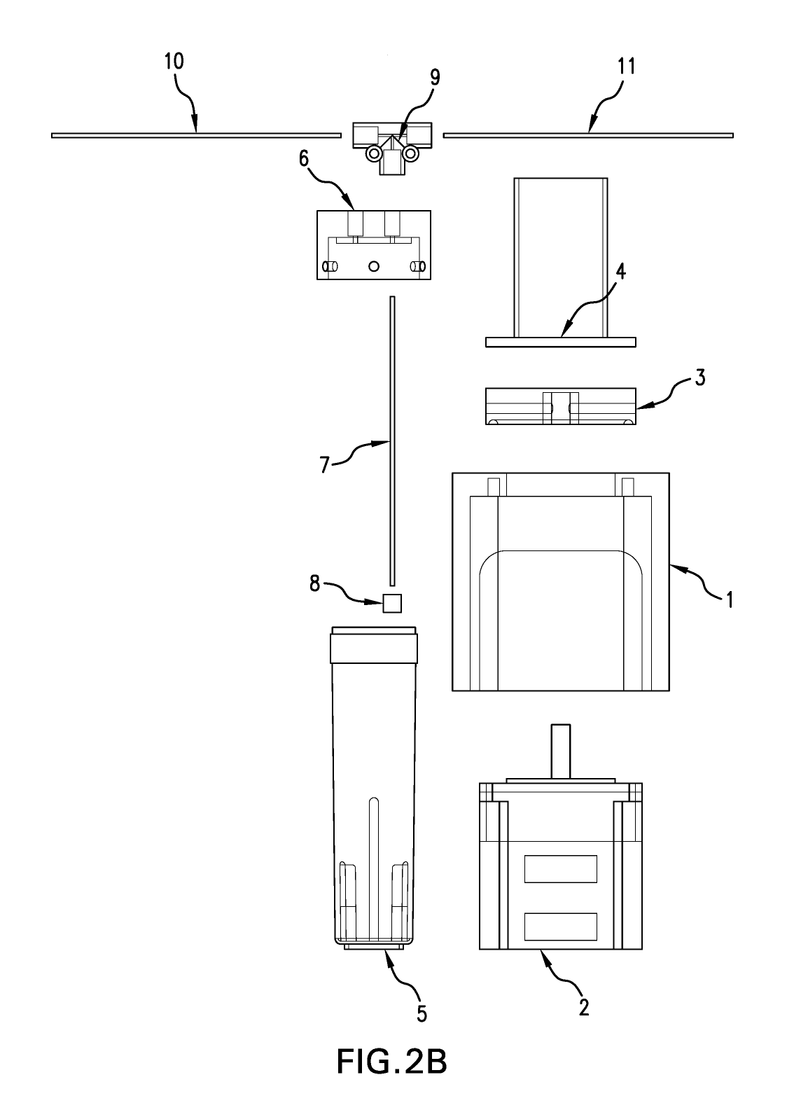

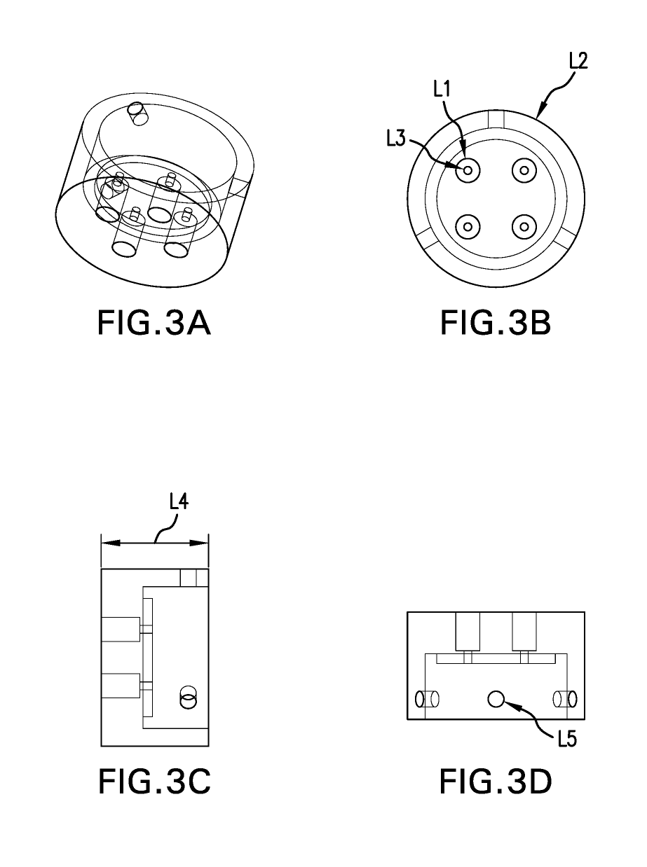

FIG. 2A depicts a side view of the central compartment. A motor 2 and optional motor cover 1 are coupled to a tube holder 4 through a magnetic coupling 3. The tube holder 4 has a magnet for attaching to the magnetic coupling 3. The culture tube 5 is supported by the tube holder 4. The motor 2 allows for the bi-directional oscillation of the culture tube 5. Media is delivered through an inlet tube 10 to a T connector 9 to which a dip tube 7 is attached and inserted through the tube cap 6 into the culture tube 5. Spent media is pumped out through the dip tube 7 through the T connector 9 and out through the outlet tube 11 to waste. At the end of the dip tube is a filter 8 which prevents the cells/microcarriers from being pumped into the outlet tube 11 and into waste. The filter is chosen based on a size cutoff, typically 75 microns.

### Cell Processing

Cells from any non-adherent cell line of interest or any adherent cell line of interest are obtained from standard tissue culture flasks. Approximately 10⁶ to 10⁷ cells (e.g., 6×10⁶ cells) are transferred to an impeller-free culture tube having baffles to allow gentle mixing of the contents without moving parts while avoiding unidirectional shear. Such a tube is commercially available and sold as the LeviTube™ (Global Cell Solutions). For adherent cells, microcarriers are added (preferably as a slurry with an equal volume of culture media).

The central compartment can be modified to grow essentially any suitable adherent mammalian cell through the use of microcarriers made of any substrate on which adherent cells can attach and grow. Microcarriers are small spheres with surfaces designed to achieve high yield monolayers of anchorage-dependent (or adherent) cells in culture. Suitable substrates include cross-linked polystyrene, microbeads and plastic flakes. The substrate can be a microcarrier composed of collagen-, fibronectin-, pronectin- or gelatin-coated microbeads or uncoated microbeads.

Commercially available microcarriers include PolyGEM microcarriers (Global Cell Solutions).

Microcarriers are usually suspended in culture media by gentle stirring. Six characteristics contribute to the optimum microcarrier: (1) suitable surface properties for cell attachment, spreading growth, and (for certain applications) genetic transformation; (2) density only slightly greater than the surrounding media (i.e., 1.030-1.045 g/mL); (3) narrow size distribution within the range of 100-230 μm diameter; (4) optical clarity for observing cell behavior; (5) non-toxic; and (6) some degree of compressibility to minimize cell damage when particles collide. Microcarriers may be categorized into four groups by their surface properties and applications: (a) cationic functional group microcarriers (e.g., microcarriers coupled to cationic amino acids or lipids), (b) neutral functional group microcarriers, (c) anionic functional group microcarriers (e.g., microcarriers coupled with nucleic acids), and (d) microcarriers coated with extracellular matrix materials (e.g., collagen, fibronectin, vitronectin, laminin, and proteoglycans).

The critical cell-to-microcarrier inoculation ratio necessary to obtain a negligible proportion of empty microcarriers in culture is calculated. An inoculation ratio of >7 ensures that <5% of microcarriers are unoccupied, and maximizes the use of available surface area. Inoculation ratios when microcarriers are employed in the central compartment can range from 7 to several hundred depending on the specific application. The highest inoculation ratios are used for very high density cell culture applications. For gene transfection of adherent cells the inoculation ratio would be markedly lower.

To allow cells to attach to microcarriers, the culture tube, containing cells and microcarriers, is mixed briefly (e.g., 1-2 minutes) then allowed to settle for approximately 0.5-1 hour. This cycle is repeated for up to 4 hours until cells have attached to the microcarrier and then the cells are mixed continuously until ready for use. The mixing is preferably done two-way and can be done by hand or in any suitable apparatus for cell culture mixing which may include a programable device such as the BioWiggler (Global Cell Solutions).

Thus, in a particular embodiment, the present invention utilizes a small device, the BioWiggler™ (Global Cell Solutions, Charlottesville, Va.) which enables cells to be plated on microcarriers in a standard incubator without the need for specialized equipment to control temperature and CO₂ levels. This device is particularly suitable for use with LeviTubes™ (Global Cell Solutions) which are 50 ml suspension culture tubes that have small baffles on the inner walls which keep cells or microcarriers suspended in media by simple oscillation of the LeviTubes™.

Prior to use in the HulaCell PK-PD device, the cells/microcarriers are allowed to settle, spent media and unattached cells are removed and fresh culture media is added to the tube.

### Applications of the HulaCell PK-PD System

The systems of the invention are thus useful for in vitro drug studies particularly for determining the relationship between pharmacokinetic parameters (which can be set using the system) and desired pharmacodynamic parameters with respect to a particular drug or agent and a particular target cell, thus enabling a method to determine a dosing regimen to best achieve a desired pharmacodynamic effect on the target cell of interest. See Lin et al., PLoS Computational Biology 7(2):el001073 (2011). Therapeutic agents to be tested include, but are not limited to, antiviral agents, antimicrobial agents (e.g., antibiotics), antineoplastic agents, antiarrhythmic drugs, cardiovascular drugs (e.g., antihypertensive drugs), antiinflammatory agents, immunosuppressive agents, immunostimulatory drugs, drugs used in the test of hyperlipoproteinemias, and asthma, drugs acting on the central nervous system (CNS), hormones, hormone antagonists, vitamins, hematopoietic agents, anticoagulants, anti-parasitic agents (i.e., anti-protozoan parasitic agents, e.g., melarsoprol), thrombolytic and antiplatelet drugs. In a preferred embodiment, the systems of the invention are used to determine the relationship between pharmacokinetics and pharmacodynamics of therapeutic agents in order to optimize dosing regimens.

In certain embodiments of the invention, an amount of one or more drugs are added to the system via the access point in the first connection line for a period of up to 21 days (e.g., 1 to 21 days). In one preferred embodiment, the cells are incubated overnight at 37° C. However, those skilled in the art will recognize that the incubation time and temperature can be adjusted to match the clinical treatment protocols.

In the methods of the invention, the amount of the one or more drugs can be constant. Alternatively, the one or more drugs can be added by infusion and/or periodically. Moreover, the amount of the one or more drugs can be varied by adjusting the flow of the cell culture medium, the dosing schedule, or both. For example, the one or more drugs can be administered in multiple short-term infusions, in intermittent infusions, or in any combination thereof.

Likewise, drug concentration/time profiles that are expected or have been obtained in human or animal studies can be simulated and the effects on cells of changing drug concentrations as anticipated in vivo may be observed in the system. Together with mathematical modeling, these in vitro paradigms can support the optimization of design of subsequent animal and human studies, thereby saving time and expense. Moreover, because a wider range of drug concentrations can be studied in vitro than in animal models, selection of appropriate concentrations for in vivo studies may become more efficient.

Any of the methods disclosed herein can also include the step of harvesting one or more of: cell culture medium; metabolites or drugs; or any combination thereof from the access point in the second connection line for analysis. For example, such analysis can include, but is not limited to, one or more pharmacodynamic studies selected from: flow cytometry for cell cycle arrest or subpopulations of cells; microarray analysis for gene profiles; RT-PCR for gene expression; Western blot for anti-apoptotic or pro-apoptotic proteins; and/or direct cell counts.

Additionally, the methods of the invention can also include the step of harvesting cells (from the microcarrier, if used) for analysis. By way of non-limiting example, the analysis of the harvested cells can be selected from measurements of cell cycling, analysis of gene expression, and/or subpopulations of drug sensitive or resistant cells. Those skilled in the art will recognize that mathematical modeling can also be used to consider the entire time course of cell counts in response to multiple concentrations of the one or more drugs.

In a particularly preferred aspect, the systems of the invention are used to study and determine the relationship between various pharmacokinetic parameters and a desired pharmacodynamic effect on cells of interest, i.e., the pharmacokinetic-pharmacodynamic (“PK-PD”) relationship of a drug to a target cell.

Pharmacokinetics concerns the absorption, distribution, biotransformation (metabolism, if it exists), and the elimination of drugs, i.e., what the body does to the drug. Relevant pharmacokinetic terms include Cₘₐₓ, the peak (maximum) concentration; AUC, the area under the drug concentration-time curve; Tₘₐₓ, the time of peak drug concentration; Cl (clearance rate), the measure of the body's ability to eliminate the drug (volume per unit time); volume of distribution, the ratio of the amount of drug in the body to the concentration of the drug in the blood; t_{1/2}, drug half-life and Cₘᵢₙ, the concentration before the next dose is administered, or any combination thereof.

Once the target cells of the central compartment are exposed to one or more therapeutic agents or drugs under a particular set of pharmacokinetic parameters, pharmacodynamic parameters can be determined. Pharmacodynamics is the study of the relationship between drug concentration and intensity of pharmacological effect, i.e., what the drug does to a target cell. Relevant pharmacodynamic parameters include essentially any characteristic or phenotype of a cell or the surrounding medium which can be assayed, e.g., growth, death, change in expression of a gene or protein, induced or reduced expression of a protein, marker, or enzymatic activity, appearance or disappearance of a metabolic product or intermediate product. More traditional pharmacodynamic parameters that could be determined could include Eₘₐₓ, the maximum effect, ED₅₀, the dose which produces 50% of the maximum effect, and EC₅₀, the concentration observed at half the maximal effect, the level of growth, the level of cell death, the change in the expression of a gene, the change in the level of a metabolic product of the cell or of the drug itself, or any combination thereof. Cells and medium from the central compartment of the bioreactor can be directly sampled via one or more ports. Methods and techniques for measuring the above pharmacodynamic parameters will depend entirely on which parameters are sought, the particular cells of interest, the drug or therapeutic agent of interest, etc. One of ordinary skill in the art will be able to measure and/or determine such parameters without undue experimentation as such methods are conventional in the art.

Mathematical modeling is utilized (see Drusano et al., 2004. Nat Rev Microbiol 2:289-300; Bilello et al., 1994. Antimicrob Agents Chemother 38: 1386-91) to increase the amount of information gained from the reported experiments. By considering the entire time course of cell counts in response to multiple concentrations of experimental drug(s) and the control treatment simultaneously, more insight can be gained into the dose-response relationship and the mechanism of action of each drug or drug combination. Also, mechanism-based models such as those described herein are more useful in making predictions, e.g., for other dosage regimens, than empirical growth models are.

Depending on the cell and drug/therapeutic agent of interest and the particular pharmacokinetic parameters used, the present invention allows one to measure various pharmacodynamic parameters, in a time-dependent fashion, including the expression of pathogen and/or cellular products, e.g., DNA, RNA (mRNA), and other products. In this system, one has access to the target cells where the drug/agent will produce its effect, so one can measure pathogen and/or cellular DNA, RNA, and proteins, such as core proteins, receptors, antigens (e.g., autoantigens, alloantigens, heteroantigens), antibodies, and enzymes.

Thus, the present invention is also directed to methods of using the in vitro cell culture system described herein, the method comprising the steps of: a) seeding 1×10⁶ to 5×10⁷ cells in the central compartment; b) placing the central compartment in connection with the in vitro culture system described herein; c) exposing the cultured cells to one or more drugs; d) removing samples of culture medium for testing; and/or removing samples of the cells for testing. The methods of the invention can be used in situations where the amount of the one or more drugs is added by one or more bolus infusions, is added periodically, is constant over time, or is added at varying rates.

The present invention is also directed to methods of determining the relationship between a pharmacokinetic parameter of a therapeutic agent and a pharmacodynamic effect of the agent on target cells, comprising: (a) growing the cells in the central compartment of the in vitro culture system described herein; (b) contacting the cells with at least one therapeutic agent; (c) adjusting the in vitro culture system to establish one or more pharmacokinetic parameters; (d) measuring at least one pharmacodynamic effect of the agent on target cells; and (e) determining a result.

The systems of the invention can also be used for dose-finding for novel drugs. The model facilitates the transition in drug development from late preclinical testing into FDA Phase I and early Phase II clinical trials. It may be used to segregate/congregate patients with specific responses for clinical trials.

In combination with pharmacodynamic modeling and by including information about the expected pharmacokinetics of a drug, the cell culture system permits study of the dose-response relationships of therapeutic agents over a very wide concentration range in vitro, and can support translation from in vitro models to animal models and human clinical trials.

### Modification of the PK/PD System for Evaluation of Bacterial Cells.

Those skilled in the art will recognize that the system as described here is intended for application to non-adherent and adherent mammalian cells. However, technical modifications in the system can be utilized in order to evaluate bacterial cells.

One skilled in the art would readily appreciate that the present invention is well adapted to carry out the objects and obtain the ends and advantages mentioned, as well as those inherent therein. The methods and compositions described herein, as presently representative of preferred embodiments, are exemplary and are not intended as limitations on the scope of the invention. Changes therein and other uses will occur to those skilled in the art, which are encompassed within the spirit of the invention, are defined by the scope of the claims.

### EXAMPLES

### Example 1: Protocols for HulaCell Assembly, Use and Sampling

The HulaCell system was designed based on using LeviTubes™ (Global Cell Solutions) as the central compartment for cell growth and media exchange. The HulaCell cap (“HulaCap”) was a custom-made plastic cap with an O-ring designed to fit and seal the top of a LeviTube™. The HulaCap has three threaded extended point setscrews that fit into the slots on the LeviTube™ and lock the HulaCap onto the top of the LeviTube™ in the same manner that the LeviTube™ vented cap locks. The thicker plastic of the HulaCell cap has four (4) threaded ports on the top that allow ¼-28 IDEX connectors to be threaded. Each HulaCell cap was initially set up with an inlet/outlet assembly, an infusion line, a gas vent and a sample port.

Magnetic LeviTube™ adapters for use with BioWiggler™ (Global Cell Solutions) were used to support the LeviTube™ and allow operation with a magnetic platform adapted to fit to four precision motors (Animatics Smartmotor Cat. No. SM2315D) coupled to a power supply (Animatics Cat. No. PS24V8A).

The media inlet/outlet (I/O) assembly was composed of a filter, a 5-10 μm porous stainless steel filter or a small cylinder of polyvinylidene fluoride (˜¾ cm in diameter x˜1 cm thick) cut from a 90 μm porous sheet, pressed onto the end of ˜6″ of HPLC (“High Performance Liquid Chromatography”) tubing ( 1/16″ OD (outside diameter); 0.010″ ID (inside diameter)) or a 16 gauge blunt aspirating needle. The stainless steel tubing or needle was threaded through the HulaCell cap and secured with a PEEK nut and ferrule (IDEX) so the bottom of the filter was at the desired depth of the central compartment volume. The end of the stainless steel tubing above the HulaCell cap was connected with a PEEK nut and ferrule to the center of a PEEK “T” connector (IDEX). It is possible to incorporate additional in-line filters (hollow filber filtration tip) into the media inlet/outlet assembly between the HulaCell cap and the PEEK “T” connector using appropriate Luer fittings. The arms of the “T” fitting were connected to two pieces of 1/16″ ID PFA tubing, ˜6′ long, using ¼-28 male connectors on the “T” fitting. These pieces of tubing were designated the ‘inlet tubing’ and ‘outlet tubing’. The other end of the inlet tubing, opposite the “T” fitting, was connected through a female Luer to 1/16″ fitting (IDEX) to the outlet end of a double check valve (Value Plastics) on the end of the media delivery syringe. The other end of the outlet tubing, opposite the “T” fitting, was connected through a male Luer to 1/16″ fitting (IDEX) to the side arm of a double check valve (Value Plastics) on the end of the waste removal syringe. The side arm of the double check valve on the media delivery syringe was connected through a male Luer to 1/16″ fitting to the media reservoir bottle cap. The outlet end of the double check valve on the waste removal syringe was connected through a female Luer to 1/16″ fitting to the waste reservoir bottle cap. (Connections to the media and waste reservoir bottle caps are described below)

The infusion line was prepared by cutting a piece of 1/16″ ID PFA tubing long enough to reach from the infusion pump to a biosafety cabinet. A female Luer to 1/16″ tubing connector (IDEX) was attached to one end and the other end is threaded with a PEEK nut and ferrule (IDEX) ˜3″ from the end. The end with the PEEK nut passes through one of the ports on the HulaCell cap and was secured with the PEEK fitting so the end of the tubing is below the surface of the media. Having the infusion line below the surface of the media ensures that low flow-rate infusions were introduced into the LeviTube at a constant rate rather than drop-wise.

To provide a gas vent in the central compartment, ˜2″ of 3/16″ Tygon tubing was attached onto a P-619 (male Luer to threaded, no lock) fitting and secured in one of the HulaCell cap ports. The end of the tygon tubing was fitted around an Acrodisc™ syringe filter (Cole-Palmer)

To provide an optional sample port, a piece of 1/16″ ID PFA tubing about 8″ long was fitted with a female Luer to 1/16″ tubing connector on one end and threaded with a PEEK nut and ferrule ˜3″ from the other end. The end with the PEEK nut was passed through one of the ports on the HulaCell cap and secured so the end of the sample line is below the media level. The PEEK nut was tightened to secure the sample line.

Any unused ports in the HulaCell cap were sealed with a threaded plug (IDEX P-311).

Each HulaCell Media bottle cap (California Plastics Cat. No. WF-38-4KIT with custom made insert containing 4×¼″-28 ports) was set up with sufficient tubing on the under-side of the cap to reach to the bottom of the media reservoir (typically a 500 ml Nalgene™ bottle; Cat. No. 2015-0500) and enough tubing on the outside of the cap to reach from the syringe pumps to a near-by biosafety cabinet (in the event the media reservoir needs to be accessed during operation). The media cap was also equipped with a gas vent to prevent vacuum lock during operation.

To create the media line, a piece of 1/16″ tubing was cut long enough to reach from the infusion pump to a near-by biosafety cabinet with an additional 12″. A male Luer to 1/16″ tubing was attached to one end and a PEEK nut and ferrule threaded ˜10″ from the other end. The end with the PEEK nut was passed through one of the ports on the media bottle cap and secured in the port so the end of the tubing reaches to the bottom of the media reservoir. The PEEK nut was tightened to secure the media line at the desired depth. The media reservoir was connected through the male to 1/16″ tubing to the side arm of the double check valve on the media delivery syringe.

To create the gas vent in the media bottle cap, ˜2″ of 3/16″ Tygon tubing was attached onto a P-619 (male Luer to threaded) fitting and passed into one of the Media bottle cap ports. Before use, an Acrodisc™ syringe filter (Cole-Parmer) was attached to the end of the tygon tubing.

The waste bottle cap was set up very similarly to the media bottle cap. The tubing need not reach to the bottom of the reservoir but should extend 2 to 3″ below the bottom of the waste bottle cap. Also, the connection at the end of the tubing must be a female Luer to 1/16″ tubing fitting. The waste cap was also equipped with a gas vent to prevent back pressure during operation.

To create the waste line, a piece of 1/16″ tubing was cut to the appropriate length. A female Luer to 1/16″ tubing connector was attached to one end and a PEEK nut and ferrule was threaded ˜3″ from the other end. The end with the PEEK nut was threaded through one of the ports on the waste bottle cap and the waste line secured 2 or 3″ below the bottom of the waste bottle cap. The PEEK nut was tightened to secure the waste line. The waste bottle is connected through the female Luer to 1/16″ tubing to the outlet arm of the double check valve on the waste removal syringe.

To create the gas vent in the waste media cap, ˜2″ of 3/16″ Tygon® tubing was attached onto a P-619 (male Luer to threaded) fitting and threaded into one of the media bottle cap ports. Before use, an Acrodisc™ syringe filter (Cole-Parmer) was attached to the end of the tygon tubing.

6-channel reciprocating syringe pumps (New Era Cat. No. NE-1600) were used to deliver fresh media and remove waste. Before the media delivery and waste removal syringes were loaded onto the reciprocating pump, the syringe plungers were removed from the barrels and the plungers were passed through a zinc-plated fender washer (¼″×1¼″ or ½″×1¼″; Home Depot) then the plungers were replaced in the syringe barrels. The washer inner diameters must be small enough to prevent the syringe plungers from passing through the hole. Each 6-channel pump has 2×1 ml media delivery syringes with double check valves and 2×3 ml waste removal syringes with double check valves loaded on it. The 3 ml syringes were loaded on one side of the pump and the 1 ml syringes were loaded on the other. The center two positions were left empty. The reciprocating arm was pushed to the HOME position so all syringes contained as little media as possible (min volume) before starting the pumps.

The infusion lines were connected to infusion syringes controlled by a single-channel infusion pump (New Era Cat. No. NE-1000) containing the drug of interest. If the drug solution is stable, multiple doses of drug can be prepared and loaded into a syringe large enough (e.g., 25-30 ml Syringe (VWR Cat. No. 53548-024) to hold all of the drug solution required for a multi-dose study. This syringe was attached to the infusion line in the same manner the other connections were made.

Before sterilization the Luer end fittings on all of the 1/16″ tubing lines were capped with appropriate male or female Luer end caps (Cole-Palmer) and the long pieces of tubing were coiled into a loose coil and secured with a small piece of tape. The tygon tubing of the vent ports was loosely wrapped with a small piece of aluminum foil. Each part was placed in a separate sterilization pouch (Cardinal Health) and the tubing coils were secured away from the free tubing ends by taping the coil to the inside of the sterilization pouch with a small piece of tape.

All parts which are not autoclavable were sterilized with 70% ethanol (check valves, Acrodisc™ filters, hollow fiber filter tips). All connections not described specifically can be done by the skilled biomedical engineer.

Plating conditions must be worked out for each cell line used in the HulaCell. As an example, a description for Huh7 GT 1B (clone 16) cells is given:

Huh7 cells were seeded onto Collagen-coated Poly-GEM™ microcarrier beads (VWR) using a 4 hour inoculation protocol on Day −1 as follows:

Cells were tyrpsinized from two T175 flasks and counted. A NucloeoCounter® (Chemometec, Allerod, Denmark) was used to determine cell count. The volume of cell suspension to contain ˜6×10⁶ Huh7 cells was calculated.

5 ml of a ˜50% (v/v) slurry of collagen coated Poly-GEM™ microcarriers in water, sterilized by autoclaving, was pelleted by centrifugation. The sterilization fluid was aspirated off and the packed microcarriers were washed once with 10 ml of Huh7 cell media containing 10% FBS (Fetal Bovine Serum). The media was removed and the microcarriers were resuspended in an equal volume of media to make a 50% slurry.

In each of 4 LeviTubes™ 0.5 ml of washed Poly-GEM™ microcarrier 50% slurry (0.25 ml packed microcarriers) was combined with 6×10⁶ Huh7 cells in 9.5 ml of media. The BioWiggler™ was used to run the following plating protocol: [Stir 1 minute at 60 oscillations per minute, Settle 40 minutes] for 4 hours. Then, the tubes were stirred continuously at 60 oscillations per minute.

Validation experiments using Huh7 cells containing GT1B HCV replicon were typically run at flow rates of 0.1 ml/min with one drug or a combination of drugs and dosed once to three times per day. At these flow rates ˜450 ml of media was required to run a 3 day experiment. If faster flow rates or longer treatment times are required, larger media reservoirs may be used or additional media may be added to the system during operation.

Before attaching the LeviTube™ with cells to the HulaCell, the microcarriers were washed one time with media to remove unattached cells from the central compartment. The plated cells and microcarriers were diluted to 40 ml with media and left stationary until the microcarriers settled (˜10 min). Approximately 30 ml of media, and unattached cells, was aspirated from the LeviTube™, being careful not to disturb the layer of microcarriers on the bottom. The LeviTube™ was then refilled with fresh media before attaching the HulaCell cap.

Preliminary studies with HCV media and cells determined that 0.5 ml of homogeneous sample from the HulaCell contained sufficient media for PK determination by mass spectroscopy analysis and sufficient live cellular material for PD determination by replicon (RT-PCR) assay. PD samples were removed from the central compartment of the HulaCell either by removing the HulaCap and pipetting or through the sample port in the HulaCap using a 1 ml syringe.

The oscillation speeds (1×360® oscillation per second) of the precision pumps were programed prior to initiation of the experiment. Optimal oscillation speed for the cell line of interest must be determined separately.

### Example 2: Inhibition of HCV Replicon Cell Line 5-2 (Genotype 1B) with a HCV NS3/4 Protease Inhibitor in HulaCell

Three QD infusions of the HCV NS3/4 protease inhibitor, (1aR,5S,8S,10R,22aR)-N-[(1R,2S)-1-[(Cyclopropylsulfonamido)carbonyl]-2-ethenylcyclopropyl]-14-methoxy-5-(2-methylpropan-2-yl)-3,6-dioxo-1,1a,3,4,5,6,9,10,18,19,20,21,22,22a-tetradecahydro-8H-7,10-methanocyclopropa[18,19][1,10,3,6]dioxadiazacyclononadecino[11,12-b]quinoxaline-8-carboxamide (“Compound”; See U.S. Pat. No. 7,973,040) were tested in the Hula Cell using the in vitro PK-PD software program and PK was monitored by collecting samples at multiple time point during each infusion. In addition, microcarrier beads were also collected daily to measure changes in viral load by RT-PCR Taqman analysis of cell lysates.

A frozen vial of HCV genotype 1B replicon cells (5-2 cells which contain genotype 1B replicon and a Luciferase reporter) was thawed and seeded into one T-75 flask. At confluency, the flask was trypsinized and cells resuspended to 6×10⁶ cells in 9.5 ml DMEM containing 10% FBS and 0.5 mg/ml G418 in a 50 ml LeviTube™. 0.5 ml of collagen-coated microcarrier bead slurry mixture (50% V/V) was added to the 9.5 ml of cells and attachment was performed using BioWiggler™ microcarrier loading program (RPM=60; 1 min mixing; 40 min settle (repeat 6x=4 hr total)). Continuous mixing at 60 rpm was performed overnight. The next morning, 30 ml of fresh media was added to the culture. The microcarriers and attached cells were allowed to settle before removing 30 ml of media with any unattached cells from the LeviTube™. Afterward, 30 ml of fresh media was added back to the LeviTube™ to make the final volume 40 ml. Then the LeviTube™ with 40 ml media, microcarrier beads and attached HCV genotype 1B replicon cells, was transferred to the HulaCell system and the pumps were started at a flow rate of 0.1 ml/min.

Dosing with Compound was initiated once the system was operational and targeted AUC=16.8 nM, Cₘₐₓ=2.5 nM, Cₘᵢₙ=0 nM and T_{1/2}=4.7 hrs. The PK/PD program was set to infuse 3 ml of 50 nM Compound at 0.1 ml/min for 30 minutes into the central compartment at T=0 min, T=1440 min and T=2880 min where T=0 min=T₀=the start of the first QD infusion. Samples were collected once daily to determine cell count, and at multiple time points to determine RNA content (PD) and drug concentration (PK).

Sampling from the HulaCell was performed as follows: The LeviTube™ comprising the central compartment of the HulaCell was removed from the 37° C. incubator and placed into a laminar flow hood. The HulaCell cap assembly was removed and tube was manually mixed by oscillating for 1 min before 200 μl or 500 μl samples of media and cell-coated microcarriers were pipetted into 1 ml microfuge tubes.

Once a day, one sample was used to determine cell number using a NucleoCounter® (Chemometec). Samples were prepared as follows: 200 μl of Buffer A (Chemometec solution 10—cell lysis buffer) was added directly to a 200 μl sample in a microfuge tube. The tube was mixed by flicking the tube several times and incubating at room temperature for 5 min. 200 μl of Buffer B (Chemometec solution 11—stabilization buffer) was then directly added to the tube and mixed. The sample was loaded into a Nucleocounter Via1-Cassette™ (Chemometec; Catalog No 941-0012), and counted using the “cell aggregates buffer A+B” program.

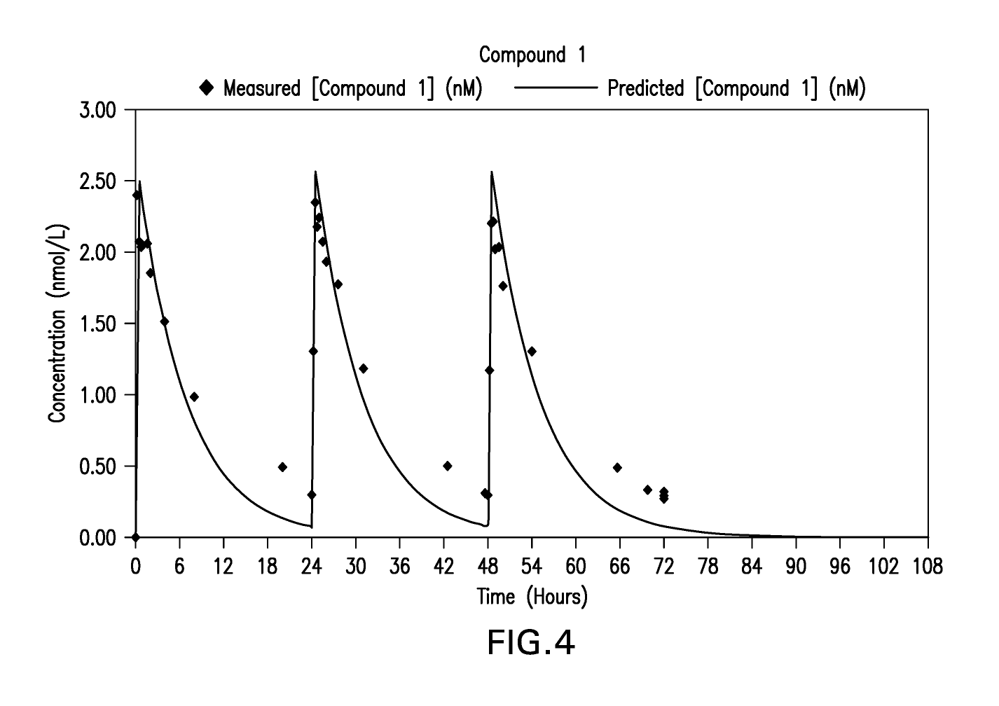

PK/PD samples were collected on Day 0, Day 1, Day 2, and Day 3 directly from the 50 ml LeviTube™ at specified time points. For collection, the computer program was paused to temporarily stop the media delivery pumps and to delay the clock which counts down to the next drug infusion. The magnetic motor turned off and the 50 ml HulaCell tube with attached tubing was removed from the 37° C. incubator and placed in the laminar flow hood. The HulaCell cap was removed and 500 μl of samples was removed from the central compartment while mixing the tube by oscillating. Samples were transferred to 1.5 ml Eppendorf tubes. Microcarrier beads were removed from samples using a desktop microfuge (30 sec spin). The microcarriers and bound cells were prepared as below for PD analysis. The supernatant was transferred to 96-well sample block and sent for PK analysis. Compound concentration in the PK samples was determined by Mass Spectroscopy analysis. Results are shown in FIG. 4.

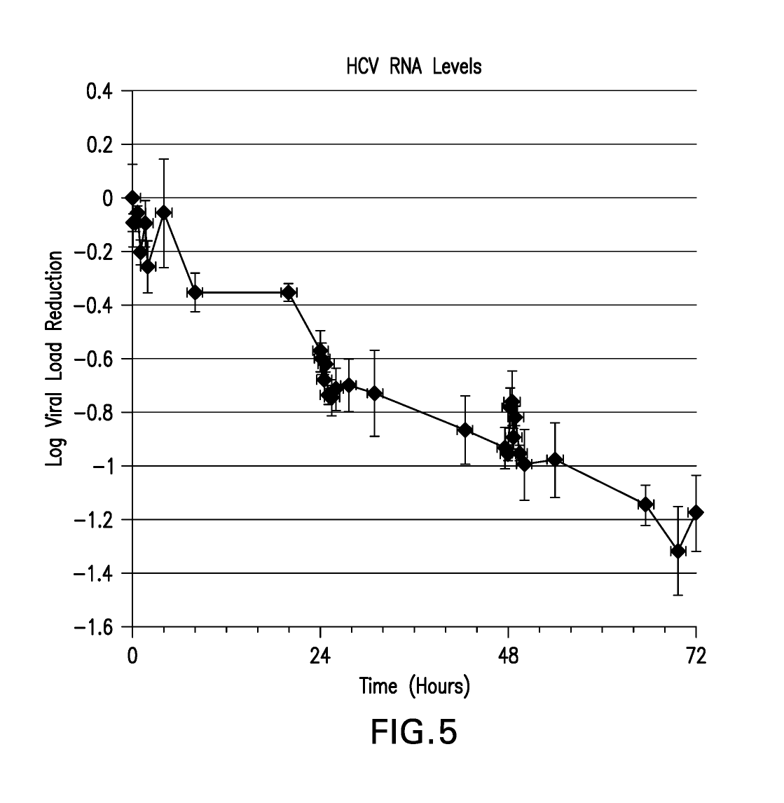

Samples for PD determination (viral load content by RT-PCR) were washed once with 1 ml PBS (w/out Mg²⁺ and Ca²⁺). Then 200 μl Ambion Cell lysis reagent was added to tubes, vortexed, heated at 70° C. for 5 min and stored at −80° C. until all PD samples were collected. Viral RNA content of the cells was determined by quantitative PCR. HCV 1B RNA concentration was determined relative to GAPDH (Glyceraldehyde 3-phosphate dehydrogenase) RNA for each sample and the reduction in HCV 1B RNA is reported relative to Day 0 (pretreatment) content. Results are shown in FIG. 5.

### Example 3: Determination of the PK Driver for Compound Using HulaCell PK-PD Platform

A 4-tube HulaCell system was validated by comparing the results for the Compound PK driver experiments, previously performed in the BelloCell® (data not shown), with the results obtained from dose escalation and dose fractionation studies performed using the HulaCell system.

Cell plating onto microcarriers was performed as in Example 2.

Samples for the PD assays, luminescence reporter assay and viral content by RT-PCR, were removed daily from the HulaCell at 0, 24, 48 and 72 hours as described in Example 2, 200 μl of cells and media was removed for each sample. PD samples for the RNA content assay were prepared as described in Example 2.

Luciferase assay (PD): Luciferase Assay was performed as follows: 200 μl culture supernatant containing microcarrier beads was spun in a microcentrifuge for 1 min at 12K rpm. The supernatant was removed and cells were washed 1× with PBS (w/out Mg and Ca). Pelleted microcarrier beads and cells were resuspended in 250 μl 1× luciferase lysate buffer (Promega Corp. Catalog # E501) and stored at −20° C. until all PD samples had been collected. To measure luciferase activity, samples were thawed and 25 μl of cell lysate was added to each well in white luminometer 96-well plate. 100 μl of luciferase reagent was added to each well and luminescence signal was read in a Perkin Elmer EnVision® Multilabel Reader Model 2101 at 0.1 sec/well.

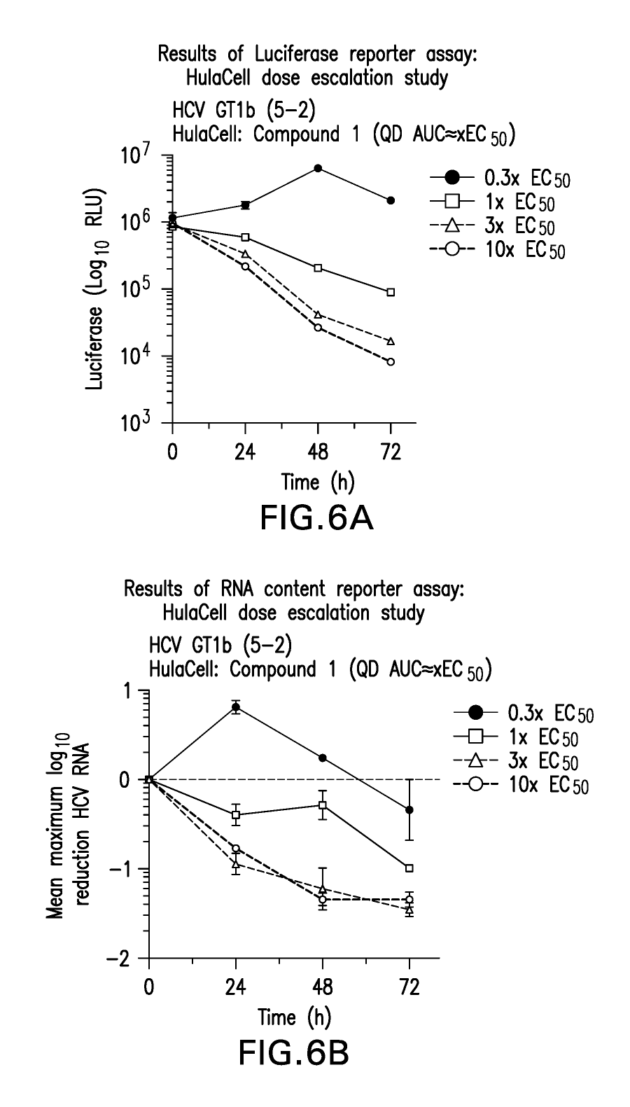

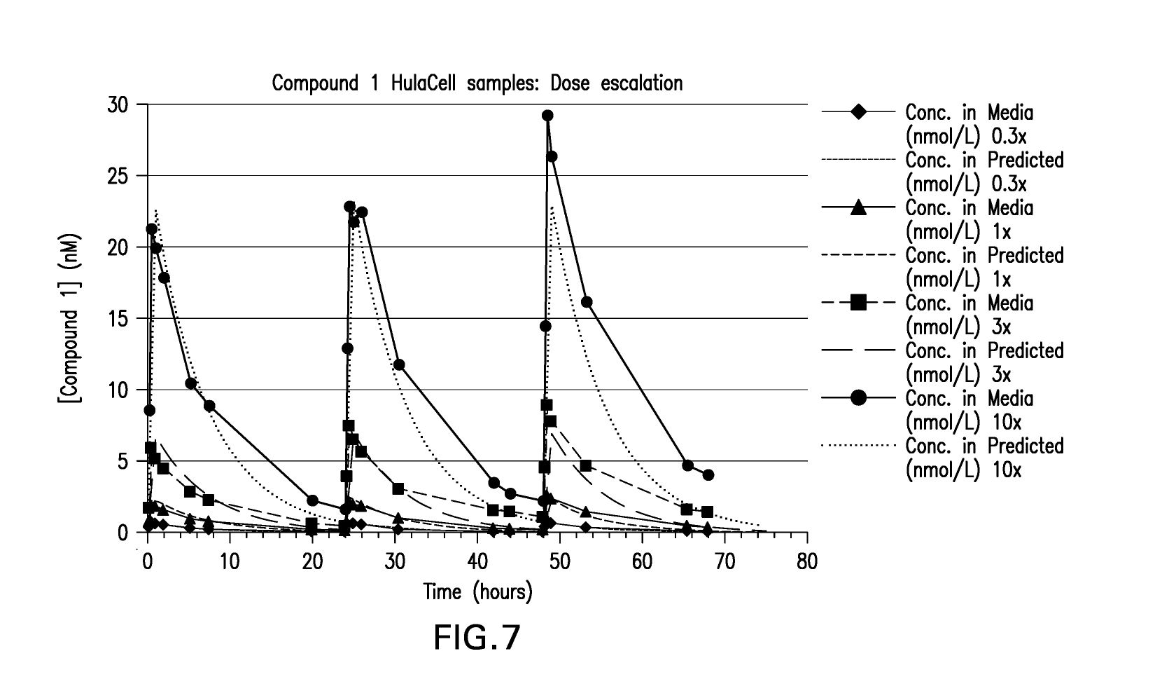

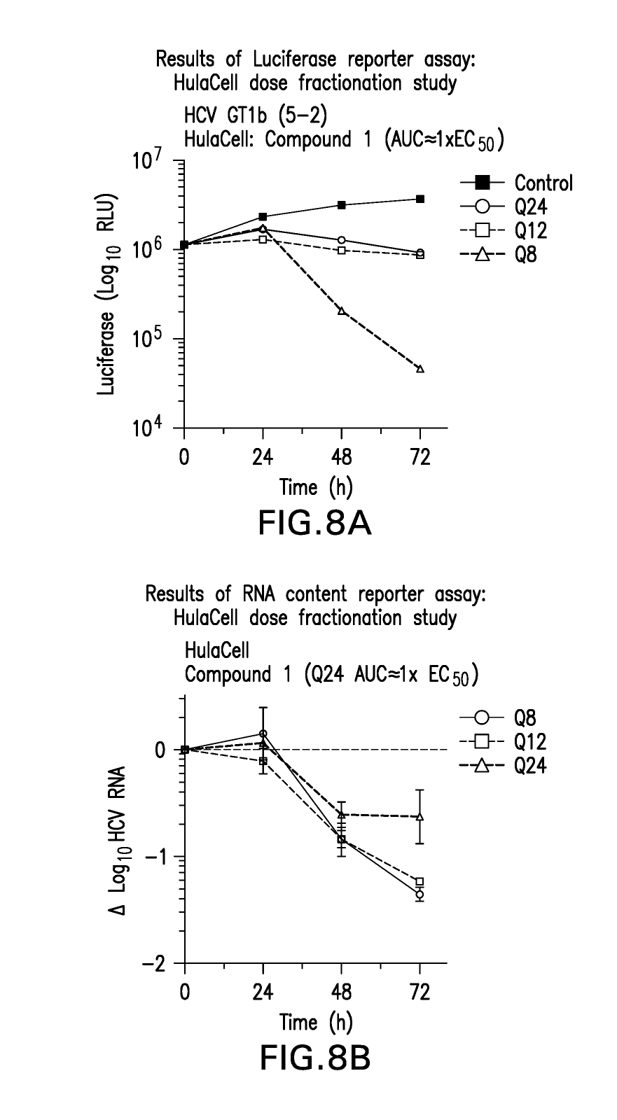

The EC₅₀ of Compound against HCV genotype 1B containing 5-2 cells was determined to be 0.7 nM in plate-based RNA content assays (data not shown). A dose escalation experiment in which HCV genotype 1B 5-2 cells were dosed QD over three days at 0.3×EC₅₀, 1×EC₅₀, 3×EC₅₀ and 10×EC₅₀ and a dose fractionation experiment in which cells were dosed QD, BID or TID to obtain a constant AUC of ˜16.8 nM were performed using the HulaCell system. Results of the reporter assays for the dose escalation experiment are shown in FIGS. 6A-B. The combined PK results of the dose escalation experiment area shown in FIG. 7. Results of the reporter assays for the dose fractionation experiment are shown in FIGS. 8A-B.

Calculated PK Driver parameters from HulaCell experiments, compared to those determined in BelloCell experiments are shown in Table 1.

TABLE 1
Comparison of Maximum Efficacy Threshold
values adjusted to free fraction

| Parameter | BelloCell | HulaCell |
|---|---|---|
| Cmin | ≥0.11 nM | ≥0.10 nM |
| % T > EC₅₀ | ≥50% | ≥84% |
| AUC₀₋₂₄ | ≥8.4 nM · hr | ≥23.7 nM · hr |

The Hula Cell was successfully used to determine the Maximum efficacy threshold values at Day 3 for Compound in the HCV genotype 1B (5-2) replicon cell line. The PK correlation curves were remarkably similar between the BelloCell® and Hula cell with the only noticeable differences being the prediction of the % T>EC₅₀ and the AUC. This system has displayed similar viral load declines versus target PK parameters as were observed in the BelloCell®.

What is claimed is:

1. An in vitro cell culture system comprising:
a) a central compartment comprising cell culture media and a filter;
b) a first syringe pump for supplying cell culture media to the central compartment, wherein the first syringe pump has a unidirectional valve and wherein the inlet line of the valve on the syringe pump is connected to the media vessel and the outlet line of the valve on the syringe pump is connected to the central compartment;
c) one or more additional syringe pumps for supplying one or more test drugs attached to the central compartment via one or more additional connection lines; and
d) a waste syringe pump used to remove waste products and drugs from the central compartment, wherein the waste syringe pump has a unidirectional valve and wherein the inlet line of the valve on the waste syringe pump is connected to the central compartment and the outlet line of the valve on the waste syringe pump is connected to the waste vessel;
wherein the central compartment can be oscillated from about 180® to about 360® in both directions around a vertical axis and the oscillations occur with a net zero oscillating periodic function.

2. The in vitro cell culture system of claim 1, further comprising a substrate for adherent cells to grow.

3. The in vitro cell culture system of claim 2, wherein the substrate is a microcarrier or plastic flake.

4. The in vitro cell culture system of claim 3, wherein the substrate is a microcarrier composed of collagen, fibronectin, pronectin or gelatin-coated or uncoated microbeads.

5. The in vitro cell culture system of claim 1, wherein the system provides a low shear cell culture environment.

6. The in vitro cell culture system of claim 5, wherein the low shear cell culture environment is provided by central compartment which is a vial having between 2 and 10 baffles along the interior side of the vial wall.

7. The in vitro cell culture system of claim 1, wherein the vial has a cap.

8. The in vitro cell culture system of claim 7, wherein the cap has a port for introducing and removing cell culture media, one or more ports for introducing one or more drugs, and a venting port.

9. The in vitro cell culture system of claim 1, wherein the filter prevents cells from entering the waste media (second connection) line.

10. The in vitro cell culture system of claim 9, wherein the filter is a porous polyvinylidene fluoride substrate with pore size less than 100 microns.

11. The in vitro cell culture system of claim 9, wherein the filter is a hollow-fiber filtration tip.

12. The in vitro cell culture system of claim 1, wherein the oscillation is achieved using a programmable motor.

13. The in vitro cell culture system of claim 1, wherein the syringe pumps are controlled by computer software.

14. A method of using the in vitro cell culture system of claim 1, the method comprising the steps of:
a) seeding 1×10⁶ to 5×10⁷ cells in the central compartment;
b) placing the central compartment in connection with the in vitro culture system of claim 1;
c) exposing the cultured cells to one or more drugs; and
d) removing samples of culture medium for testing; and
e) removing samples of the cells for testing.

15. The method of claim 14, wherein the amount of the one or more drugs is added by one or more bolus infusions.

16. The method of claim 14, wherein the amount of the one or more drugs is added periodically.

17. The method of claim 14, wherein the amount of the one or more drugs added is constant over time.

18. The method of claim 14, wherein the amount of the one or more drugs is added at varying rates.

19. The method of claim 14, further comprising using mathematical modeling to achieve one or more pharmacokinetic parameters selected from maximum concentration (Cₘₐₓ), the area under the drug concentration-time curve (AUC), the time of peak drug concentration (Tₘₐₓ), the clearance rate (Cl), the drug elimination rate (volume per unit time), the concentration before the next administered dose (Cₘᵢₙ), the drug half-life (T_{1/2}) or any combination thereof.

20. The method of claim 14, further comprising testing the cell sample for a pharmacodynamics property selected from cell viability, cell growth, cell shape, viral load, expression of a protein, post-translational modification of a protein, DNA content, modification of DNA, RNA content, expression of a lipid marker or any combination thereof.

21. A method of determining the relationship between a pharmacokinetic parameter of a test drug and a pharmacodynamic effect of the test drug on target cells, comprising: (a) growing the cells in the central compartment of the in vitro culture system of claim 1; (b) contacting the cells with at least one test drug; (c) adjusting the in vitro culture system to establish one or more pharmacokinetic parameters; (d) measuring at least one pharmacodynamic effect of the test drug on target cells; and (e) determining a result.
